(19) United States
(12) Patent Application Publication
(10) Pub. No.: US 2004/0142903 A1
(43) Pub. Date: Jul. 22, 2004

(54) Bioavailable fenofibrate compositions, methods for treating hyperlipidemia and hypercholesterolemia and processes for the preparation of such compositions

(75) Inventors: Robert A. Femia, Kinnelon, NJ (US); Oscar I. Fishkis, White Plains, NY (US); Damodar P. Patel, Elmwood Park, NJ (US); Narayan Ragunathan, West Nyack, NY (US)

Correspondence Address: THE FIRM OF KARL F ROSS, 5676 RIVERDALE AVENUE, PO BOX 900, RIVERDALE (BRONX), NY, 10471-0900, US

(73) Assignee: PAR PHARMACEUTICAL INC.

(21) Appl. No.: 10/345,882

(22) Filed: Jan. 16, 2003

(51) Int. Cl. A61K 31/724; A61K 31/194

(52) U.S. Cl. 514/58; 514/571

(57) ABSTRACT

Pharmaceutical compositions for treating hyperlipidemia or hypercholesterolemia in mammals are described which comprise:

| | | |
|---|---|---|
| (a) | fenofibrate | 5 to 35 wt. -% |
| (b) | a cyclodextrin | 4 to 30 wt. -% |
| (c) | an alkali metal or | 0.1 to 10 wt. -%; and |
| | alkaline earth metal docusate and/or | |
| | alkali metal or alkaline earth metal | |
| | lauryl sulfate | |
| (d) | a water-insoluble, wettable | 5 to 30 wt. -% |
| | inorganic carrier capable of forming a | |
| | dispersion of the fenofibrate | |
| | and a pharmaceutically acceptable | |
| | inert carrier or diluent. | |

A therapeutically effective amount of the compositions are orally administered to mammals to treat hyperlipidemia or hypercholesterolemia.

### FIELD OF THE INVENTION

This invention relates to novel fenofibrate compositions with bioavailability. The invention further relates to compositions containing fenofibrate, a cyclodextrin, an alkaline metal or alkaline earth metal docusate and/or an alkali metal or alkaline earth metal lauryl sulfate, and a water-insoluble, wettable carrier which provide fenofibrate to patients in a highly bioavailable form without the need for co-micronization of fenofibrate with any of the other ingredients.

### BACKGROUND OF THE INVENTION

Fenofibrate is a well known antihyperlipoproteinemic agent. See U.S. Pat. No. 4,058,552. Experience with oral administration of fenofibrate has shown that the bioavailability of the drug has not been as high as would be desirable. A good deal of research has been carried out over the years to obtain compositions containing fenofibrate that are orally administered to patients and which have improved bioavailability. According to U.S. Pat. No. 4,895,726 to CURTET et al compositions containing fenofibrate with improved bioavailability have been prepared in which the fenofibrate has been co-micronized in an intimate mixture with a solid surfactant such as sodium lauryl sulfate. CURTET et al expressly state that it is possible to improve the bioavailability to a significantly greater extent than that which would be achieved either by adding a surfactant, or by micronizing the fenofibrate on its own, or by intimately mixing the separately micronized fenofibrate and surfactant. Such a fenofibrate-containing composition is currently available on the market under the mark TRICOR^{R}.

Among the compositions actually exemplified in CURTET et al, all such compositions contain alpha-lactose monohydrate, a well known hydrosoluble carrier. The alpha-lactose monohydrate is added to the co-micronizate of fenofibrate and solid surfactant.

Co-micronization of the fenofibrate and the solid surfactant is a difficult, expensive and time-consuming process and it would be desirable to obtain pharmaceutical compositions suitable for oral administration containing fenofibrate as the active ingredient that have a good bioavailability and yet do not require co-micronization with a solid surfactant.

A disclosure of fenofibrate-containing compositions with high bioavailability may be found in U.S. Pat. Nos. 6,074,670 and 6,277,405 to STAMM et al. The STAMM et al compositions contain fenofibrate in micronized form and contain a hydrosoluble carrier such as lactose and a surfactant such as sodium lauryl sulfate, and a hydrophilic polymer such as a polyvinylpyrrolidone. Co-micronization of the fenofibrate with the surfactant is disclosed as optional. Nonetheless the STAMM et al compositions all require both a hydrosoluble carrier and a hydrophilic polymer as well as a series of complex steps in order to obtain the composition with high bioavailability.

In Acta Pharm. 46 (1996) 131 to 136, experiments were carried out on micronized fenofibrate with a view to determining solubilization of and enhancing dissolution of the sparingly soluble fenofibrate. Sodium lauryl sulfate and docusate sodium were each used as surfactants to improve the solubility of micronized fenofibrate in water. It was found that both sodium lauryl sulfate and docusate sodium significantly accelerated the dissolution rate of fenofibrate, the magnitude of the effects of these surfactants increasing with the amount added, but not in a linear way. Addition of surfactants exceeding 10 mg per 200 mg micronized fenofibrate dosage unit were thus considered not interesting. The authors concluded that sodium lauryl sulfate and docusate sodium were equally potent dissolution enhancers for micronized fenofibrate over a wide concentration range.

According to S.T.P. Pharma Sciences 7(2) 174 to 181 (1997) inclusion complexes were formed with β-cyclodextrin or hydroxypropyl-β-cyclodextrin as “host” molecule and fenofibrate as the “guest” molecule. The fenofibrate is disclosed as finely powdered although no particle size is given and no specific mention is made of micronization. The purpose of forming the β-cyclodextrin inclusion complexes of fenofibrate was to overcome the poor solubility of fenofibrate in water. The authors hoped that improving the water-solubility of the fenofibrate by forming its β-cyclodextrin inclusion complexes would possibly improve bioavailability. The reference discloses forming an inclusion complex with fenofibrate as “guest” molecule and β-cyclodextrin as “host” molecule using either dispersion, kneading or spray-drying to prepare the inclusion complex. Spray-drying did prove to be the best way to prepare a stable inclusion complex with improved water-solubility. It is disclosed that fenofibrate and β-cyclodextrin in a 1 to 2 molar ratio gives the most stable inclusion complex with the best water-solubility.

The problem with formation of the β-cyclodextrin inclusion complexes of fenofibrate is that formation of the inclusion complex is difficult, time-consuming and may not be commercially feasible. The reference process to prepare the inclusion complexes preferably employs fenofibrate and at least a double molar quantity of β-cyclodextrin dissolved at 40° C. in the lowest volume of ethanol necessary to obtain a solution under stirring for 30 minutes. This may be due to poor water solubility of β-cyclodextrin. In order to facilitate complex formation the amount of the ethanol may be increased. After that the solution is spray-dried under the following conditions: feed rate 10 ml/min; inlet temperature 95° C., outlet temperature 65° C.; pressure 5 bar; throughput of drying air 35 cu m/hour. The collected powders are stored under a vacuum in a desiccator for 3 days and then analyzed.

When the ethanol amount utilized is large, however, the complex formation by the β-cyclodextrin is reduced. Also, if solvent evaporation takes place too fast during the spray-drying, the fenofibrate may crystallize and may not form the complex and there is no improvement in solubility and bioavailability of the fenofibrate.

It would be especially advantageous if a composition containing fenofibrate that is highly bioavailable could be prepared much more easily and consistently without the need to employ ethanol in large amount as a solvent to form the composition.

### OBJECTS OF THE INVENTION

It is an object of the invention to obtain new pharmaceutical compositions containing fenofibrate with a high bioavailability and which are highly suitable for oral administration to a patient in need of therapy for reducing the level of lipids in the blood.

It is a further object of the invention to provide pharmaceutical compositions containing fenofibrate with high bioavailability along with other ingredients which facilitate the bioavailability of the micronized fenofibrate without the need to co-micronize the fenofibrate with any of the other ingredients in the composition and without the need to apply the fenofibrate suspended in a surfactant solution to a hydrosoluble carrier.

### SUMMARY OF THE INVENTION

We have found that our new pharmaceutical compositions containing fenofibrate have high bioavailability. Our new pharmaceutical compositions comprise a core having the following ingredients expressed as weight %:

| | | |
|---|---|---|
| (a) | fenofibrate | 5 to 35 wt -% |
| (b) | cyclodextrin | 4 to 30 wt -% |
| (c) | alkali metal or | 0.1 to 10 wt -%; and |
| | alkaline earth metal docusate and/or | |
| | alkali metal or alkaline earth metal | |
| | lauryl sulfate | |
| (d) | a water-insoluble, | 5 to 30 wt -% |
| | wettable inorganic carrier capable of | |
| | forming a dispersion of the fenofibrate | |

The core may be coated with any pharmaceutically acceptable inert, moisture barrier coating suitable to protect a pharmaceutically active ingredient from the environment. Preferred coatings include Opadry grade or any suitable film coating.

The pharmaceutical compositions are prepared by mixing preferably in a high-shear mixer the four abovementioned ingredients to form a powder mixture. Next a suitable solvent which preferably includes water or a lower alkanol such as methanol, ethanol, n-propanol or isopropanol is added to the powder mixture to form a wet granulate mixture. The wet granulate mixture is then dried in a fluid bed dryer or a tray dryer oven at 50 to 100° C. to obtain a dry granulate which is milled to a particle size of about 1 mm or less and sifted through a mesh screen to remove any particles that are too large. Glidants such as colloidal silica and tableting agents such as magnesium stearate are added with disintegrants such as SSG (Sodium Starch Glycolate) and the milled granulate is then compressed into tablets. Then a thin moisture barrier coating is applied to the entire surface of the tablet.

The fenofibrate when micronized preferably has a particle size of from about 0.1 to about 40 μm, preferably 0.1 to 20 μm.

In one alternative a solution in water of an alkali metal or alkaline earth metal docusate (which can be granulated with sodium benzoate in an 85:15 weight ratio, if desired) preferably of an alkali metal and/or alkaline earth metal lauryl sulfate is sprayed onto a powder mixture of fenofibrate, a cyclodextrin, and a water-insoluble, wettable inorganic carrier capable of forming a dispersion of the fenofibrate to form a wet granulate. Once the wet granulate is formed the drying, milling and tablet-forming steps are the same as described hereinabove.

In another alternative water is sprayed onto a powder mixture of fenofibrate, an alkali metal or alkaline earth metal docusate and/or an alkali metal or alkaline earth metal lauryl sulfate, cyclodextrin, and a water-insoluble, wettable inorganic carrier capable of forming a dispersion of the fenofibrate to form a wet granulate. Once the wet granulate is formed the drying, milling and tablet-forming steps are the same as described hereinabove.

In another alternative a suspension of micronized fenofibrate, and an alkali metal or alkaline earth metal docusate and/or an alkali metal or alkaline earth metal lauryl sulfate, in water is sprayed onto a powder mixture of the cyclodextrin, the water-insoluble, wettable inorganic carrier capable of forming a dispersion of the fenofibrate, and a diluent to form a wet granulate. Once the wet granulate is formed the drying, milling and tablet-forming steps are the same as described hereinabove.

In another alternative a suspension of micronized fenofibrate, an alkali metal or alkaline earth metal docusate and/or an alkali metal or alkaline earth metal lauryl sulfate, and a cyclodextrin in water is sprayed onto a powder mixture of the water-insoluble, wettable inorganic carrier capable of forming a dispersion of the fenofibrate, and a diluent to form a wet granulate. Once the wet granulate is formed the drying, milling and tablet-forming steps are the same as described hereinabove.

In another alternative fenofibrate and a cyclodextrin are co-milled to a mean particle size of not more than 40 μm and added to water, then an alkali metal or alkaline earth metal docusate and/or an alkali metal or alkaline earth metal lauryl sulfate, is blended in and the water-insoluble, wettable inorganic carrier capable of forming a dispersion of the fenofibrate, and a diluent in the form of a powder are added to form a wet granulate. Once the wet granulate is formed the drying, milling and tablet-forming steps are the same as described hereinabove.

In another alternative fenofibrate, a cyclodextrin, and an alkali metal or alkaline earth metal docusate preferably an alkali metal or alkaline earth metal lauryl sulfate, are co-milled to a mean particle size of not more than 40 μm, and added to water, then a water-insoluble, wettable inorganic carrier capable of forming a dispersion of the fenofibrate, and a diluent in the form of a powder is added to form a wet granulate. Once the wet granulate is formed the drying, milling and tablet-forming steps are the same as described hereinabove.

A preferred feature of the invention includes forming a colloidal suspension of the fenofibrate. The particle size of the fenofibrate in this case is very small, preferably about 0.1 to 0.4 μm or 100 to 400 nanometers, with a more preferred range of 0.1 to 1.2 μm.

Examples of cyclodextrins include α-cyclodextrin, β-cyclodextrin, γ-cyclodextrin, and substituted β-cyclodextrins such as 2,6-di-O-methyl- β-cyclodextrin or hydroxypropyl-β-cyclodextrin. It is preferred that the cyclodextrin be a poorly water-soluble cyclodextrin such as β-cyclodextrin.

The water-insoluble, wettable inorganic carrier capable of forming a dispersion of the fenofibrate is preferably soluble in an acidic solution and is most preferably dibasic calcium phosphate dihydrate, preferably in the form of a milled powder.

In order to form the new pharmaceutical compositions additional ingredients to those mentioned hereinabove may be added. Examples of such additional ingredients include diluents such as microcrystalline cellulose, disintegrants such as sodium starch glycolate, pharmaceutical binding agents such as dibasic calcium phosphate dihydrate, inert glidants such as colloidal silicon dioxide, tablet-forming aids such as magnesium stearate.

A critical part of the invention is the inclusion of an alkali metal or alkaline earth metal docusate such as docusate sodium and/or an alkali metal or alkaline earth metal lauryl sulfate such as sodium lauryl sulfate in the compositions according to the invention. Also contemplated within the scope of the invention is a surfactant which comprises a mixture of the alkali metal or alkaline earth metal docusate and the alkali metal or alkaline earth metal lauryl sulfate in a proportion of 1:100 to 100:1.

We have found that the combination of the micronized fenofibrate active ingredient, the cyclodextrin, especially a poorly water-soluble cyclodextrin such as β-cyclodextrin, and an alkali metal or alkaline earth metal docusate and/or an alkali metal or an alkaline earth metal lauryl sulfate together form a suspension or solution which upon spray drying or wet granulation permits formation of a tablet which enables a large amount of the fenofibrate to be placed into solution and therefore to be highly bioavailable to a patient taking such a composition by mouth.

Another aspect of the present invention is a method for the treatment of hyperlipidemia or hypercholesterolemia comprising the step of orally administering to a mammalian subject, including a human, a therapeutically effective amount of the new pharmaceutical composition according to the present invention.

### EXAMPLE 1

### Manufacturing Procedure

In a high shear mixer fenofibrate micronized to a mean particle size of not more than 20 microns, β-cyclodextrin, and docusate sodium were mixed with microcrystalline cellulose, sodium starch glycolate, and dibasic calcium phosphate dihydrate as a milled powder to form a powder mixture. Ethanol purified under the USP was added to the powder mixture. The wet granulate mixture was dried in a fluid bed dryer or a tray dryer oven at 50° C. The resulting dry granulate of high purity was then milled to a particle size of about 0.7 mm and sifted through a 0.8 mm sieve to remove particles that are too large. Colloidal silica, sodium starch glycolate and magnesium stearate NF was then added to the milled granulate. The final blend was then compressed into oval-shaped tablets on a rotary tableting machine.

A thin coating of Opadry was applied over the entire surface area of each tablet.

The following specific compositions presented in Table I were prepared according to the procedure set forth in Example 1 containing either 54, 107 or 160 mg of the pharmaceutically active ingredient micronized fenofibrate:

TABLE I
Comparison Between strengths (54 mg, 107 mg and
160 mg). Each Fenofibrate Tablet 54 mg, 107 mg
and 160 mg contains the following:

| | 54 mg | 107 mg | 107 mg | 160 mg |
|---|---|---|---|---|
| | mg/ | mg/ | (bisected) | mg/ |
| Strength - Ingredients | tablet | tablet | mg/tablet | tablet |
| Fenofibrate, Micronised | 54.00 | 107.00 | 107.00 | 160.00 |
| Microcrystalline Cellulose, | 50.00 | 99.07 | 99.07 | 148.15 |
| NF (Avicel PH 101) | | | | |
| Betadex, NF (Beta Cyclodextrin, | 45.00 | 89.17 | 89.17 | 133.33 |
| C. Cavitron #82900) | | | | |
| Sodium Starch Glycolate, | 10.00 | 19.82 | 19.82 | 29.63 |
| NY (Explotab) | | | | |
| Dibasic Calcium Phosphate | 32.30 | 64.00 | 64.00 | 95.70 |
| Dihydrate, USP, Milled Powder | | | | |
| Docusate Sodium, USP with | 2.70 | 5.35 | 5.35 | 8.00 |
| Sodium Benzoate, | | | | |
| NF (DSS Granular) | | | | |
| Solvent, USP* | — | — | — | — |
| Microcrystalline Cellulose, NF | 30.00 | 59.44 | 59.44 | 89.89 |
| (Avicel PH 102) | | | | |
| Sodium Starch Glycolate, | 10.00 | 19.82 | 19.82 | 28.63 |
| NF (Explotab) | | | | |
| Colloidal Silicon Dioxide, | 2.00 | 3.96 | 3.96 | 5.93 |
| NF (Aerosil 200) | | | | |
| Magnesium Stearate, NF | 2.00 | 3.96 | 3.96 | 5.93 |
| Core Weight, | 238.00 | 471.59 | 471.59 | 705.19 |
| Opadry Yellow YS-1-12625 | 7.00 | — | — | — |
| Opadry White YS-1-7003 | — | 12.00 | 12.00 | 15.00 |
| Purified Water, USP* | — | — | — | — |
| Coated Tablet Weight, | 245.00 | 483.59 | 483.59 | 720.19 |

*Solvent, USP does not appear in the final product. (the solvent in general can be ethanol, water, isopropanol or methanol)

### EXAMPLE 2

The same procedures and reaction conditions as employed in Example 1 are employed in Example 2 except that a solution of docusate sodium in water was sprayed onto a powder mixture of the micronized fenofibrate, β-cyclodextrin, microcrystalline cellulose, sodium starch glycolate, and dibasic calcium phosphate dihydrate to form a wet granulate. Once again tablets were formed and a thin coating of Opadry was applied over the entire surface area of each tablet.

### EXAMPLE 3

The same procedures and reaction conditions as employed in Example 1 are employed in Example 3 except that water was sprayed onto a powder mixture of the micronized fenofibrate, docusate sodium, β-cyclodextrin, microcrystalline cellulose, sodium starch glycolate, and dibasic calcium phosphate dihydrate to form a wet granulate. Once again tablets were formed and a thin coating of Opadry was applied over the entire surface area of each tablet.

### EXAMPLE 4

The same procedures and reaction conditions as employed in Example 1 are employed in Example 4 except that a suspension of micronized fenofibrate and docusate sodium in water was sprayed onto a powder mixture of β-cyclodextrin, microcrystalline cellulose, sodium starch glycolate, and dibasic calcium phosphate dihydrate to form a wet granulate. Once again tablets were formed and a thin coating of Opadry was applied over the entire surface area of each tablet.

### EXAMPLE 5

The same procedures and reaction conditions as employed in Example 1 are employed in Example 5 except that a suspension of micronized fenofibrate, docusate sodium, and β-cyclodextrin in water was sprayed onto a powder mixture of microcrystalline cellulose, sodium starch glycolate, and dibasic calcium phosphate dihydrate to form a wet granulate. Once again tablets were formed and a thin coating of Opadry was applied over the entire surface area of each tablet.

### EXAMPLE 6

The same procedures and reaction conditions as employed in Example 1 are employed in Example 6 except that fenofibrate and β-cyclodextrin are co-milled to a mean particle size of not more than 40 μm and added to water, then a mixture of docusate sodium and sodium lauryl sulfate (1:1 by weight) is blended in and a powder mixture of microcrystalline cellulose, sodium starch glycolate, and dibasic calcium phosphate dihydrate is added to form a wet granulate. Once again tablets were formed and a thin coating of Opadry was applied over the entire surface area of each tablet.

### EXAMPLE 7

The same procedures and reaction conditions as employed in Example 1 are employed in Example 7 except that fenofibrate, β-cyclodextrin, and docusate sodium are co-milled to a mean particle size of not more than 40 μm and added to water, then a powder mixture of microcrystalline cellulose, sodium starch glycolate, and dibasic calcium phosphate dihydrate is added to form a wet granulate. Once again tablets were formed and a thin coating of Opadry was applied over the entire surface area of each tablet.

In any of the Examples 1 through 7 where water is called for as solvent, a lower alkanol may be substituted for the water and where a lower alkanol is called for as solvent, water may be substituted for the lower alkanol.

### BRIEF DESCRIPTION OF THE DRAWING

FIG. 1 is a set of two curves plotting the blood plasma concentration in ng/ml of the active metabolite fenofibric acid at intervals from 0.5 to 96 hours following administration of micronized fenofibrate to 38 healthy volunteer patients. One curve shows administration of the micronized fenofibrate according to the present invention and the other curve shows administration of fenofibrate using the prior art composition TRICOR^{R}.

FIG. 2 is a set of two curves plotting the same data as shown in FIG. 1. However, in FIG. 2 the blood plasma concentration is shown as the natural log rather than expressed in ng/ml.

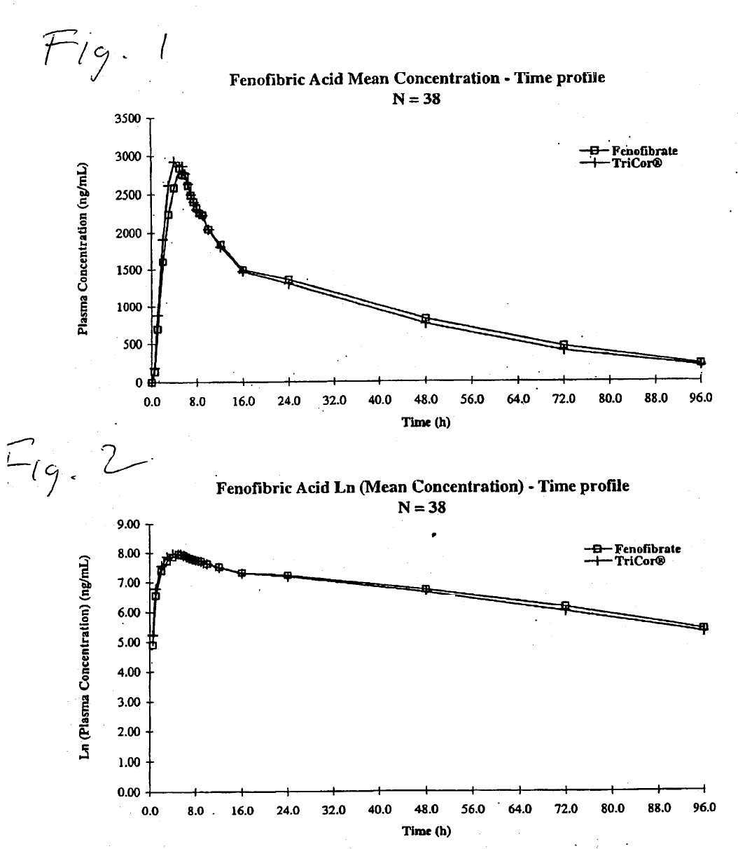

### RESULTS

Study of Bioavailability of the Compositions According to the Present Invention and the Prior art TRICOR^{R} Composition

A test of bioavailability on healthy volunteers was carried out.

The following compositions were tested:

(a) a composition prepared according to Example 1 of the invention as a tablet containing 160 mg of micronized fenofibrate; and

(b) TRICOR^{R} prior art composition also containing 160 mg of micronized fenofibrate.

The study was carried out on 38 healthy volunteers receiving a single dose of fenofibrate, with a minimum 6 days rest between administrations. The plasma samples for pharmacokinetic analysis were collected after each administration at the following times: 0.5 h; 1 h; 2 h; 3 h; 4 h; 5 h; 6 h; 8 h; 10 h; 12 h; 24 h; 36 h; 48 h; 72 h; and 96 h following administration of the medicament. The fenofibric acid (active metabolite of fenofibrate) content in the plasma was measured for each sample. The results obtained are given in Tables II and III below:

### SUMMARY OF RESULTS

### FENOFIBRIC ACID

### N=38

TABLE II
Pharmacokinetic Parameters

| | Test (Fenofibrate (A)) | | Reference (TriCor ® (B)) | |
|---|---|---|---|---|
| Parameters | Mean ± SD | CV (%) | Mean ± SD | CV (%) |
| AUC₀₋ₜ (ng h/mL) | 92212.10 ± 30999.67 | 33.62 | 88149.81 ± 26774.64 | 30.37 |
| AUC_{0-inf} (ng h/mL) | 106497.84 ± 45844.09 | 43.05 | 100821.08 ± 39392.74 | 39.07 |
| Cₘₐₓ (ng/mL) | 3116.89 ± 1066.16 | 34.21 | 3170.61 ± 1128.66 | 35.60 |
| Tₘₐₓ (h) | 5.22 ± 1.42 | 27.14 | 5.08 ± 1.61 | 31.76 |
| Kₑₗ (h⁻¹) | 0.0291 ± 0.0120 | 41.18 | 0.0295 ± 0.0108 | 36.63 |
| T_{1/2el} (h) | 28.70 ± 14.77 | 51.47 | 27.77 ± 14.75 | 53.13 |

TABLE III
Fenofibrate (A) vs TriCor ® (B)

| | AUC₀₋ₜ | AUC_{0-inf} | Cₘₐₓ |
|---|---|---|---|
| Ratio¹ | 104.09% | 104.74% | 99.52% |
| 90% Geometric | 98.41% to | 96.41% to | 91.50% to |
| C.I.² | 110.11% | 113.80% | 108.24% |
| Intra-Subject CV | 14.56% | 21.63% | 21.91% |

¹Calculated using least-squares means according to the formula: e^{(Fenofibrate (A)-TriCore ®(B))} × 100
²90% Geometric Confidence Interval using ln-transformed data
Cmax maximum plasma concentration
Tmax time to reach Cmax
T1/2el: plasma half life
AUC 0 to t: area under the curve from 0 to t
AUC 0 to inf area under the curve from 0 to infinity

The results show that the levels in the blood of the active metabolite fenofibric acid are comparable for the compositions according to the present invention and for TRICOR^{R}. These data show therefore that the bioavailability of both the present and prior art compositions is about the same. Since the present invention does not require comicronization of fenofibrate and a surfactant, an expensive and time-consuming process, in order to obtain the compositions, there is a significant advantage obtained according to the present compositions.

What is claimed is:

1. A pharmaceutical composition for treating hyperlipidemia or hypercholesterolemia which comprises:

| | | |
|---|---|---|
| (a) | fenofibrate | 5 to 35 wt. -% |
| (b) | a cyclodextrin | 4 to 30 wt -% |
| (c) | alkali metal or | 0.1 to 10 wt -%; and |
| | alkaline earth metal docusate and/or | |
| | alkali metal or alkaline earth metal | |
| | lauryl sulfate | |
| (d) | a water-insoluble, wettable | 5 to 30 wt -% |
| | inorganic carrier capable of forming a | |
| | dispersion of the fenofibrate | |
| | and a pharmaceutically acceptable | |
| | inert carrier or diluent. | |

2. The pharmaceutical composition defined in claim 1 wherein the cyclodextrin is a water-insoluble cyclodextrin.

3. The pharmaceutical composition defined in claim 2 wherein the water-insoluble cyclodextrin is β-cyclodextrin.

4. The pharmaceutical composition defined in claim 1 wherein the fenofibrate is micronized to a particle size of about 1 to 40 mμ.

5. The pharmaceutical composition defined in claim 1 which further comprises at least one tablet-forming aid, disintegrant, diluent, emulsifier or binding agent.

6. The pharmaceutical composition defined in claim 1 which further comprises a pharmaceutically acceptable coating surrounding said composition.

7. The pharmaceutical composition defined in claim 1 wherein the water-insoluble, wettable inorganic carrier capable of forming a dispersion of the fenofibrate is soluble in an acidic medium.

8. The pharmaceutical composition defined in claim 7 wherein the water-insoluble, wettable inorganic carrier capable of forming a dispersion of the fenofibrate and which is soluble in an acidic medium is dibasic calcium phosphate dihydrate.

9. A method of treating hyperlipidemia or hypercholesterolemia in a mammalian subject which comprises the step of orally administering to said subject a therapeutically effective amount of the pharmaceutical composition defined in claim 1.

10. A process for preparing a pharmaceutical composition for treating hyperlipidemia or hypercholesterolemia which comprises:

| | | |
|---|---|---|
| (a) | fenofibrate | 5 to 35 wt. -% |
| (b) | a cyclodextrin | 4 to 30 wt -% |
| (c) | alkali metal or | 0.1 to 10 wt -%; and |
| | alkaline earth metal docusate and/or | |
| | alkali metal or alkaline earth metal | |
| | lauryl sulfate | |
| (d) | a water-insoluble, wettable | 5 to 30 wt -% |
| | inorganic carrier capable of forming a | |
| | dispersion of the fenofibrate | |

and a pharmaceutically acceptable inert carrier or diluent, which comprises the steps of:
(i) preparing a wet granulate which comprises fenofibrate, a cyclodextrin, an alkali metal or alkaline earth metal docusate and/or alkali metal or alkaline earth metal lauryl sulfate, and a water-insoluble, wettable inorganic carrier capable of forming a dispersion of the fenofibrate;
(ii) drying the wet granulate at a temperature of 50 to 100° C. to obtain a dry granulate;
(iii) milling the dried granulate to a particle size of about 1 mm or less; and
(iv) adding the pharmaceutically acceptable inert carrier or diluent to obtain a final blend.

11. The process for preparing a pharmaceutical composition defined in claim 10 further comprising the step of compressing the final blend into tablets.

12. The process for preparing a pharmaceutical composition defined in claim 10 wherein according to step (a) the wet granulate is prepared by mixing micronized fenofibrate, a cyclodextrin, an alkali metal or alkaline earth metal docusate and/or alkali metal or alkaline earth metal lauryl sulfate, and a water-insoluble, wettable inorganic carrier capable of forming a dispersion of the fenofibrate to form a powder mixture and adding water or a lower alkanol to the powder mixture.

13. The process for preparing a pharmaceutical composition defined in claim 10 wherein according to step (a) the wet granulate is prepared by mixing a solution in water of an alkali metal or alkaline earth metal docusate and/or of an alkali metal or an alkaline earth metal lauryl sulfate and spraying the solution onto a powder mixture of fenofibrate, a cyclodextrin, and a water-insoluble, wettable inorganic carrier capable of forming a dispersion of the fenofibrate.

14. The process for preparing a pharmaceutical composition defined in claim 10 wherein according to step (a) the wet granulate is prepared by spraying water onto a powder mixture of fenofibrate, an alkali metal or alkaline earth metal docusate and/or an alkali metal or alkaline earth metal lauryl sulfate, cyclodextrin, and a water-insoluble, wettable inorganic carrier capable of forming a dispersion of the fenofibrate.

15. The process for preparing a pharmaceutical composition defined in claim 10 wherein according to step (a) the wet granulate is prepared by forming a suspension of micronized fenofibrate, and an alkali metal or alkaline earth metal docusate and/or an alkali metal or alkaline earth metal lauryl sulfate, in water and spraying the suspension onto a powder mixture of the cyclodextrin, the water-insoluble, wettable inorganic carrier capable of forming a dispersion of the fenofibrate.

16. The process for preparing a pharmaceutical composition defined in claim 10 wherein according to step (a) the wet granulate is prepared by forming a suspension of micronized fenofibrate, an alkali metal or alkaline earth metal docusate and/or an alkali metal or alkaline earth metal lauryl sulfate, and a cyclodextrin in water and spraying the suspension onto a powder mixture of the water-insoluble, wettable inorganic carrier capable of forming a dispersion of the fenofibrate.

17. The process for preparing a pharmaceutical composition defined in claim 10 wherein according to step (a) the wet granulate is prepared by co-milling fenofibrate and a cyclodextrin to a mean particle size of not more than 40 μm, then blending in an alkali metal or alkaline earth metal docusate and/or an alkali metal or alkaline earth metal lauryl sulfate, and the water-insoluble, wettable inorganic carrier capable of forming a dispersion of the fenofibrate.

18. The process for preparing a pharmaceutical composition defined in claim 10 wherein according to step (a) the wet granulate is prepared by co-milling fenofibrate, a cyclodextrin, and an alkali metal or alkaline earth metal docusate and/or an alkali metal or alkaline earth metal lauryl sulfate, to a mean particle size of not more than 40 μm, and then adding a water-insoluble, wettable inorganic carrier capable of forming a dispersion of the fenofibrate.

19. The process for preparing a pharmaceutical composition defined in claim 10 wherein the water-insoluble, wettable inorganic carrier capable of forming a dispersion of the fenofibrate is soluble in acid solution.

20. The process for preparing a pharmaceutical composition defined in claim 19 wherein the water-insoluble, wettable inorganic carrier capable of forming a dispersion of the fenofibrate and which is soluble in acid solution is dibasic calcium phosphate dihydrate.
